(19) United States
(12) Patent Application Publication
(10) Pub. No.: US 2012/0197146 A1
(43) Pub. Date: Aug. 2, 2012

(54) METHODS, SYSTEMS, AND MONITORING DEVICES FOR MANAGING HEMODYNAMIC PARAMETERS

(75) Inventors: Lin Tan, Shenzhen (CN); Qiang Li, Shenzhen (CN); Qinglin Tao, Shenzhen (CN)

(73) Assignee: SHENZHEN MINDRAY BIO-MEDICAL ELECTRONICS CO., LTD., Shenzhen (CN)

(21) Appl. No.: 13/326,056

(22) Filed: Dec. 14, 2011

(30) Foreign Application Priority Data
Jan. 30, 2011 (CN) CN201110033470.4

(51) Int. Cl. A61B 5/02

(52) U.S. Cl. 600/509

(57) ABSTRACT

Methods, systems, and monitoring devices for managing hemodynamic parameters are disclosed.

### CROSS-REFERENCE TO RELATED APPLICATIONS

This application claims the benefit of Chinese Patent Application No. 201110033470.4, filed Jan. 30, 2010, which is incorporated herein by reference in its entirety.

### TECHNICAL FIELD

The following disclosure relates to the field of medical monitoring.

### SUMMARY OF THE INVENTION

Disclosed herein are embodiments of methods, systems, and devices for managing hemodynamic parameters.

### BRIEF DESCRIPTION OF THE DRAWINGS

FIGS. 1 and 2 is a schematic views of hemodynamic parameter management systems;

FIG. 3 is a flow chart of a method for managing hemodynamic parameters;

FIG. 4 is an exemplary user interface of the function group map;

FIGS. 5 and 6 are map fragment views;

FIG. 7 is a schematic view of a hemodynamic parameter management system;

FIG. 8 is flow chart view of a method for managing hemodynamic parameters; and

FIG. 9 is a schematic view of a patient monitor including a hemodynamic parameter management system.

### DETAILED DESCRIPTION

Hemodynamic monitoring is extremely important in clinical anesthesia and intensive care. It is a necessary feature of major operations and critical patient rescue. In general, hemodynamic monitoring includes two types: invasive and non-invasive. Non-invasive hemodynamic monitoring acquires parameters related to cardiovascular function indirectly through the skin or mucous membranes without injuring the patient's body. Invasive hemodynamic monitoring directly measures physiological parameters by inserting catheters or sensors to heart chambers or vascular cavities from the body surface.

There are a wide variety of hemodynamic parameters, such as CCO, CO, CCI, CI, EDV, EDVI, SVR, SVRI, SV, SVI, BT, RVEF, ESV, ESVI, HR, Art, CVP, MAP, ITBV, ITBI, EVLW, EVWI, CFI, PPV, CPO, CPI and dPmx. The clinician may be able to diagnose the patient's condition by one or more of these parameters.

A monitoring device typically displays a number of major hemodynamic parameters on the screen, or provides a menu or a view that lists all the hemodynamic parameters. The clinician has to rely on his clinical experience to select a set of parameters from a large list and estimate the patient's condition using measurements of selected parameters. However, this wastes a great deal of time and effort, and the effect may not be evident. Especially during major operations or critical patient rescue, it is important to discover problems quickly and accurately from monitoring hemodynamic parameters. At present, the accuracy and speed of diagnosis depends mainly on clinician's experience. Clinicians with little experience may be at a loss when facing so many parameters, delaying treatment endangering the patient's life.

The present disclosure is directed to a method, system, and monitoring device for managing hemodynamic parameters, which can handle a large number of hemodynamic parameters according to various diseases, thereby enhancing a clinician's accuracy and speed diagnosis.

According to the present disclosure, different groups of parameters are defined, with each group including certain hemodynamic parameters that are used together to diagnose a disease or a trend of a physiological condition. In this way, the diagnosis doesn't depend as much on the clinician's experience, facilitating greater speed and accuracy of diagnosis.

As noted above, hemodynamic monitoring may involve many parameters. The clinician usually diagnoses a patient's disease or trend according to certain parameters. In one embodiment, monitoring device designers or caregivers select certain hemodynamic parameters and combine them into a group to diagnose a certain disease or reflect a trend. In this disclosure, hemodynamic parameters belonging to a certain functional group are called Selected Hemodynamic Parameters (hereinafter SHPs). A process according to the present disclosure may include finding simultaneously abnormal SHPs belonging to a functional group, comparing the abnormal condition of the SHPs with predefined rule, determining a display mode of the functional group according to the predefined rule, and then displaying a changing map of each functional group on the screen of monitoring device over time based on the determined display mode.

The abnormal situation may be of a variety of types. For example, it could be the number of simultaneously abnormal SHPs in a functional group, that is, the number of SHPs is out of range. Likewise, the abnormal situation could be the abnormal ratio of SHPs in a functional group, that is, the ratio of the abnormal SHPs to the total SHPs in a certain function group. The abnormal situation also could also be the extent to which an SHP is out of range, e.g., slightly out of range, somewhat out of range, and severely out of range. The abnormal situation could be a combination of the above listed types.

The predefined rule is related to the abnormal condition. In one embodiment, the abnormal situation is categorized into levels, each level corresponds to a display mode. For example, the predefined rule can be set as categorizing into levels according to the number of SHPs that are out of range at the same monitoring time. The rule can also be set as categorizing into levels according to the ratio of simultaneously abnormal SHPs in each functional group. The SHPs can also be assigned different weights, which may be based on the extent to which an SHP is out of range. When the SHP is abnormal, the weights may be accumulated and categorized into several levels. It will be understood by those having skill in the art that the predefined rules can also be the combination of the above listed rules or combination of other rules.

The map displays the functional group that contains various hemodynamic parameters combined to indicate a certain disease or physiological symptom. Each functional group represents one type of disease or physiological symptom. One or some of the parameter value changes will have a positive clinical significance. Therefore, even if a caregiver lacks experience, he can know a patient's situation and disease at some time from the map display mode of a functional group. The caregiver does not need to manually select and view parameters and then diagnose patient's situation and/or disease. This will enhance the caregiver's speed and accuracy of diagnosis. In addition, the map can provide information to a caregiver about a patient's condition, which is beneficial to patient treatment.

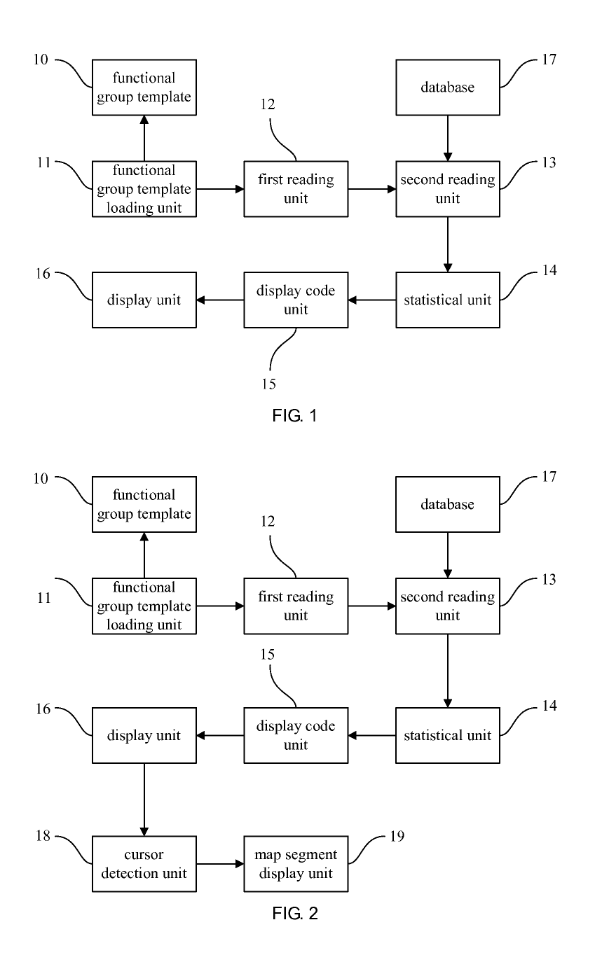

FIG. 1 illustrates a hemodynamic parameter management system. In one embodiment, the system includes a functional group template loading unit 11, a first reading unit 12, a second reading unit 13, a statistical unit 14, a display code unit 15, and a display unit 16. The functional group template loading unit 11 loads one or more predefined functional group templates 10 corresponding to certain diseases. The first reading unit 12 reads SHPs in the loaded functional group template. The first reading unit 12 notifies the second reading unit 13 of the read SHPs, and the second reading unit 13 acquires corresponding parameter information of the SHPs from a real-time monitored hemodynamic parameter database 17. The statistical unit 14 collects simultaneously abnormal SHP situation of each functional group according to information acquired by the second reading unit 13. The display code unit 15 compares the simultaneously abnormal SHP situation of each functional group with the predefined rules and determines the display mode. The display unit 16 displays the map, which may change over time, of each functional group according to the display mode determined by the display code unit 15.

As shown in FIG. 2, the hemodynamic parameter management system may further include a cursor detection unit 18 and a map segment display unit 19. When a user needs to view detailed parameter data, he can move the cursor to select some position on the function group map. The cursor detection unit 18 detects the selected position of the cursor on the function group map. The map segment display unit 19 displays the map segment at the time corresponding to the selected position. The map segment displays the SHP values in this functional group.

In another embodiment, hemodynamic parameter management system includes a map analysis unit (not shown) and a map segment display unit (not shown). The map analysis unit analyzes the map of functional groups and determines a Most Typical Time (MTT), which refers to the time when there are N abnormal SHPs in a functional group, and the time when there are N abnormal SHPs that recover to normal. In one embodiment, N is an integer greater or equal to 1. The map segment display unit displays the map segment at the above-defined MTT. The map segment contains the SHPs value in the functional group at that time.

With increased research into various diseases, there may be a requirement to change SHPs in functional groups. Therefore, the hemodynamic system may also include a functional group setup unit (not shown). The user can set hemodynamic parameters and/or functional group names through the functional group setup unit.

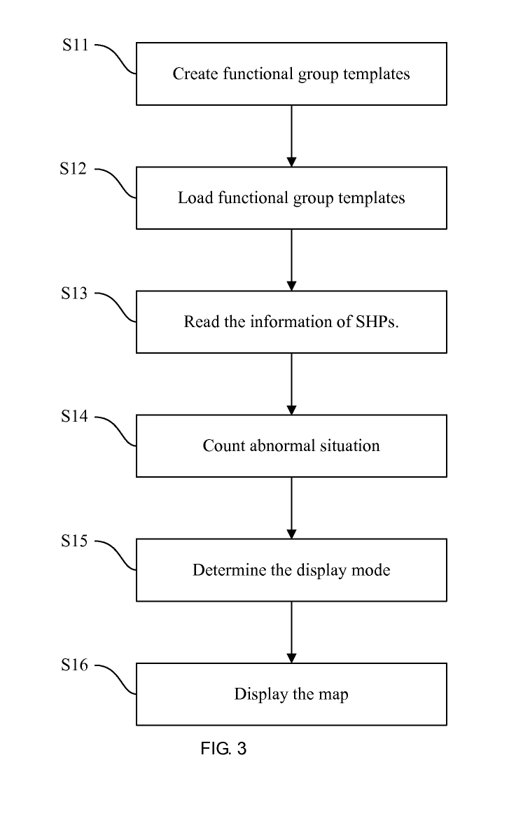

In one embodiment, as shown in FIG. 3, a hemodynamic parameter management method may include various steps as outlined below.

Step S11, Create functional group templates. The functional group templates can be created in the design phase by designers or they can be subsequently created according to user requirements. Each functional group contains one or more hemodynamic parameters to define a certain disease or physiological symptom. A functional group can be defined to include several hemodynamic parameters. Also, a hemodynamic parameter can be listed in multiple functional groups. Each functional group represents one type of disease or physiological criterion. Users can edit functional groups according to their requirements, including what and how many parameters need to be configured, renaming functional groups according to clinical customs and terminology, deleting unnecessary functional groups, and so on. Users can also set up multiple functional groups according to their requirement to evaluate the overall physiological state of patients. In addition, the trend of each functional group can be used for correlation research among functional groups, which will provide more clinical evidence. Users can adopt a disease name as the name of functional group, example, e.g., pulmonary edema, heart failure, respiratory failure, etc. From the functional group name, users can diagnose the disease and physiological trend changes of the patients.

Step S12, Load functional group templates. One or multiple predefined functional group templates may be loaded and the hemodynamic parameters (SHPs) obtained from each functional group template. For default functional group templates, the system may have already acquired the hemodynamic parameters from functional groups. In this case, it is also regarded as loading functional group templates.

Step S13, Read the information of SHPs. Information from real-time monitored hemodynamic parameters may be saved in a database in advance in various storage devices. SHP information may be read from the storage device(s). The parameter information can be the value and the monitoring time of the hemodynamic parameter, or a mark showing whether each hemodynamic parameter is abnormal.

Step S14, Count abnormal situations. Statistics may be calculated for the abnormal situations of the simultaneously selected SHPs in a functional group based on parameter information. The statistics of the abnormal situation can be calculated by following predefined rules set by users, e.g., count the number of simultaneously abnormal SHPs in each functional group, compute the ratio of simultaneously abnormal SHPs in each functional group, or count the number of SHPs beyond the normal range. If the parameter information is the value associated with monitoring time, the method may compare each SPH value with a predefined normal range to determine if the SHP is normal.

According to parameter properties, the normal range of a hemodynamic parameter can be values of a bilateral closed area or values of a unilateral closed area. If the normal range is values of a bilateral closed area, the method may compare SHP values with the normal range. There may be three results: (i) in the range, (ii) higher than the upper limit, and (iii) lower than the lower limit. If the hemodynamic parameter value is in the range, it is regarded as normal; if the value is higher than the upper limit, or lower than the lower limit, it is regarded as abnormal. The abnormal situations can be counted respectively or counted together.

If the normal range is values of a unilateral closed area, the method may compare SHP values with the normal range. There may be two results: (i) in the range or (ii) anomaly (higher than the upper limit or lower than the lower limit). The method may calculate the statistics of the anomaly. If the parameter information is the mark that indicates whether the value is in the range, the method may compare each hemodynamic parameter value with the predefined range. The mark for an abnormal situation could be of two types (above the upper limit and below the lower limit) or one type (above the upper limit or below the lower limit). If it is required to calculate the statistics of the extent of SHPs beyond the normal range, the method may compute the value.

Step S15, Determine the display mode. The method may compare the abnormal situations of the simultaneously abnormal SHPs in each functional group with predefined rules and determine the display mode of each functional group at that the time of monitoring. In one embodiment, the predefined rule is to categorize abnormal situations of SHPs into levels according to the number of abnormal SHPs, each level corresponding to a display mode. In another embodiment, the predefined rule is to categorize into levels according to the ratio of the abnormal SHPs, each level corresponding to a display mode. The latter mode requires dividing the number of total SHPs by the number of abnormal SHPs in each functional group to determine the ratio and then comparing the ratio with the predefined rules.

The display mode can be represented in many ways. For example, different colors, symbols, pictures, images, or codes may be used. When codes are used, each monitoring time of the functional groups may have the corresponding code. Different codes correspond to different states of parameters in the function group and therefore the display mode is varied. For example, suppose color coding is used. When all the parameters are within range, the display mode is green at that time. When one parameter is out of range, the display mode is yellow at that time. When more than two parameters are out of range, the display mode is red at that time. Similarly, when one parameter is higher than an upper limit, the display mode may be yellow. When one parameter is lower than the lower limit, the display mode may be red. The importance of the parameter can be through weights, with the most important parameter having higher weights, and less important parameters having lower weights. If a problem occurs, the radar diagram will indicate a state according to the variation per unit time of the parameter with a different weight.

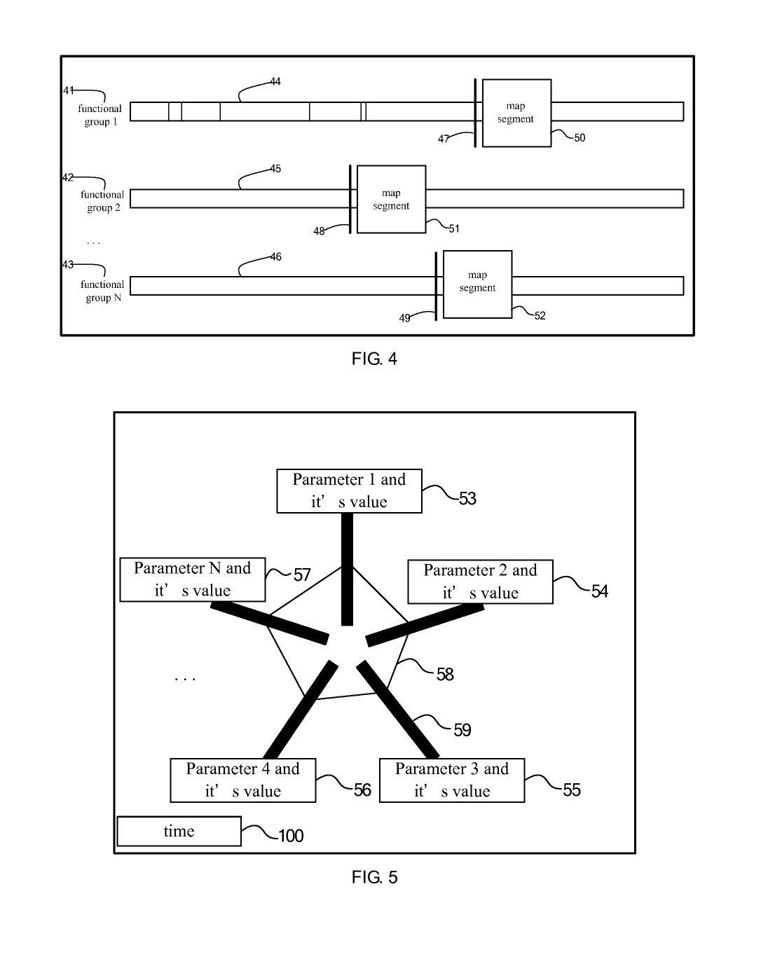

Step S16, Display the map. The method may display a changing map of each functional group according to the specified display mode. As shown in FIG. 4, there may be three functional group maps in the map display domain. In the exemplary embodiment, the three functional groups are the functional groups 41-43. The corresponding maps are the map 44-46. A linear relationship may exist between the map length and the system time of monitors: the longer the monitoring time, the longer the map length. Each time point in the map displays the image according to the corresponding display mode. The display mode corresponds to the state of parameters in a functional group at that time. Therefore, a caregiver can diagnose a patient's disease or state at that time according to the display mode. In one embodiment, when one or more functional groups are abnormal, example, e.g., the parameter is out of range, a message, such as a warning, a suggestion, or a handling measure, can be displayed at the corresponding time on the map.

In another embodiment, the parameter information is an indication of whether the parameter is normal. During collection and generation of the parameter information, the method may compare the collected physiological parameter data with the normal range in advance, and obtain the parameter information (normal or abnormal). The method may set a marker bit for each collected parameter during storage. For example, the marker for a normal parameter may be “0”, while the marker for abnormal parameter may be “1”. When reading the parameter information, reading the value in the marker bit and counting the marker bit with “1” in each functional group will obtain the number of simultaneously abnormal SHPs.

The example embodiment includes maps of three functional groups. A skilled artisan will understand that more or fewer function groups may be included.

The above functional group template creation steps can be intiated by users. If a user needs to edit the templates, he can activate the functional setup unit at any step and set the hemodynamic parameters and/or functional group names.

In another embodiment, detailed information may be displayed in the form of map segments, such as the map segments 50, 51, 52 shown in FIG. 4. The map segment displays the values of SHPs at some time in the functional group. The map segment can be used as the window for dynamically displaying functional group parameter valves. It is convenient to monitor patient real-time situations. The two display modes can be referred to at the same time in a clinical situation.

In one embodiment, the process of displaying map segments is as follows. The user locates the cursor 47, 48, 49 in the map. The system detects the cursor selected position at a certain functional group map, and then displays the map segment of the time according to the cursor selected position. When there are multiple cursor selected locations at the map, multiple map segments will be displayed.

In another embodiment, the process of displaying map segments is as follows. The method may determine the Most Typical Time (MTT) in the map by analyzing the map of a functional group and then display all map segments at the MTT. The MTT refers to the time when there are N abnormal SHPs in a functional group and the time when N abnormal SHPs recover to normality. N is an integer that is greater than or equal to 1.

The map segments can be displayed in a newly created window or in the designated area. The SHP values at that time can be displayed in the form of graph or numeric value in the map segment, such as spider vision diagram, datasheet, or histogram. As shown in FIG. 5, the map segment is displayed in the form of spider vision diagram, including the spider connection line 58, spider leg 59, the parameters and their values 53, 54, 55, 56, 57 corresponding to spider legs, and the time 100 of the map segment. The rules for drawing the spider vision diagram may include: (i) the spider legs are in the same amount of the configured parameters, (ii) all of the spider legs are of same length, (iii) the angle between the adjacent spider legs is 360°/n (n is the number of spider legs), (iv) the spider leg and the parameter measurement range has linear relationship, (v) the normal parameter range can be marked with color on the spider legs, (vi) and the connection point on a spider leg by a connection line is the current measurement value of the parameter. According to the marked normal range on the spider leg and the connection point on the connection line, a clinician will be able to know which parameter is abnormal, and whether the value is above the upper limit or below the lower limit.

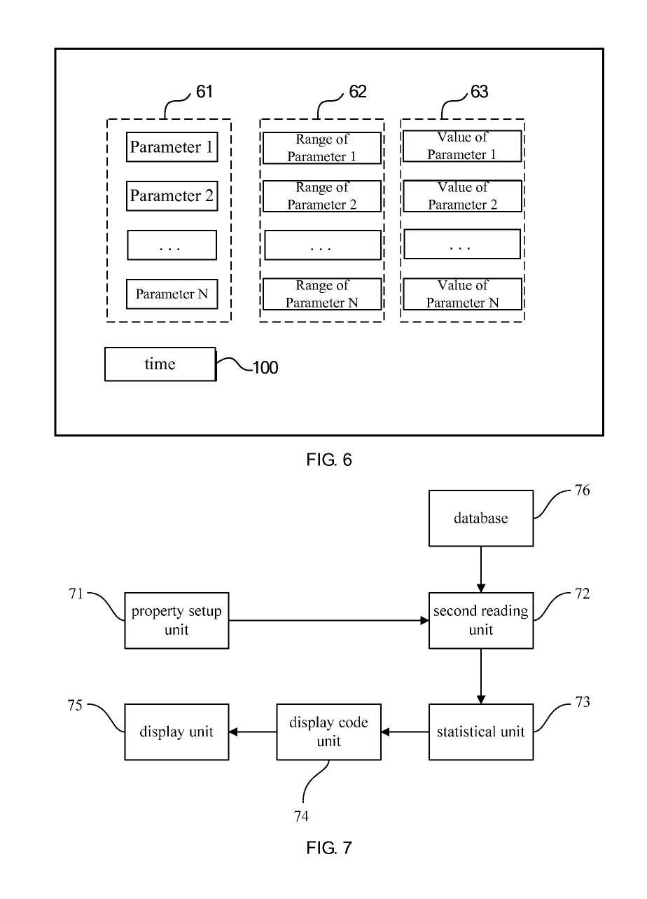

As shown in FIG. 6, the map segment is displayed in numerical form. The data sheet may include a parameter name 61, a settable parameter range 62, a parameter value 63, and a time 100 of map segments.

In various embodiments, the system can analyze multiple map segments. The analysis method can include one of the following:

1. Arrange multiple map segments of functional groups on MTT automatically or by user's configuration. The arrangement rules may include, but are not limited to, by time, by time of one or more parameters, or by alarm priority and time.

2. Superimpose multiple map segments. The system may also include a map superimposing unit that is used to superimpose map segments in a functional group on different times and then show them in one graph. For example, there may be loop connection diagrams on two or more different times in one spider vision diagram. The superimposing time can be determined by users. The superimposing display mode for map segments is more convenient to accurately analyze a patient's physiological trend.

Rules for superimposing graphical segments may include 1) superimpose graphical segments in the same functional group; 2) superimpose graphs according to the same parameter; 3) superimpose map segments by time sequence; and 4) the position of each parameter stays the same after being superimposed.

Rules for superimposing numerical segments may include 1) superimpose numeric segments in the same functional group; 2) superimpose segments with the same parameter name; 3) parameter values at the same time are combined to a corresponding parameter set.

The above embodiment provides an effective tool for medical monitors to manage patient physiological parameters according to physical sign trending in diagnosing diseases and symptoms. This management tool supports the creation of functional groups for managing diseases. The tool can provide a period of a patients' physiological trend change; it also can provide real-time diagnosis for a decision maker. This tool can efficiently manage physiological parameters, simplify the user of monitors, save medical resources, save time, and enhance diagnostic efficiency.

In another embodiment, the monitoring device designer or caregiver selects some hemodynamic parameters that can be combined to diagnose a disease and sets those parameters with the same properties, e.g., they may have the same mark. The specified hemodynamic parameter management system is shown in FIG. 7, and may include a property setup unit 71, a second reading unit 72, a statistical unit 73, a display code unit 74, and a display unit 75. The property setup unit 71 sets properties of hemodynamic parameters. The second reading unit 72 acquires parameter information of SHPs with properties from the database 76, which stores real-time hemodynamic parameter values over time. The statistical unit 73 collects the abnormal situation of SHPs with the same property at the same monitoring time according to parameter information. The display code unit 74 compares the abnormal situation of the simultaneously abnormal SHPs with same properties at the same time with predefined rules and determines the display mode of SHPs with the properties at that monitoring time. The display unit 75 takes the SHPs with the same properties as a functional group and displays the functional group map that changes over time according to the determined display mode.

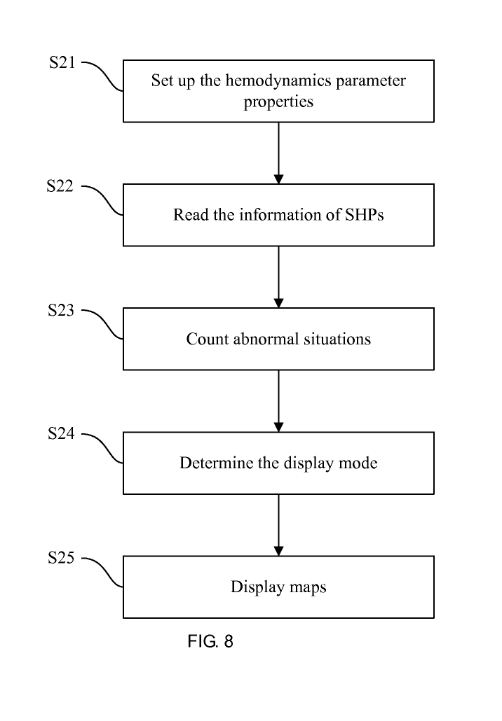

Based on the above system, a hemodynamic parameter management method may include the following steps, as shown in FIG. 8.

Step S21, set up the hemodynamics parameter properties in advance. The parameters used to represent the same disease are of same properties. Different diseases will have the parameters of different properties.

Step S22, read the information of SHPs. The method may acquire the parameter information of the SHPs with properties from the real-time hemodynamic parameter values related to time.

Step S23, count abnormal situations. The method may calculate the statistics of abnormal situations of the simultaneously abnormal SHPs with the same properties. The detailed statistical method is same as previously described.

Step S24, determine the display mode. The method may compare the abnormal situations of the simultaneously abnormal SHPs with same properties with predefined rules and determine the display mode of the parameter with those properties at that monitoring time. The method for determining the display mode may be the same as described previously.

Step S25, display maps. The method may take the SHPs with the same properties as a functional group and display the functional group map changing over time according to the determined display mode.

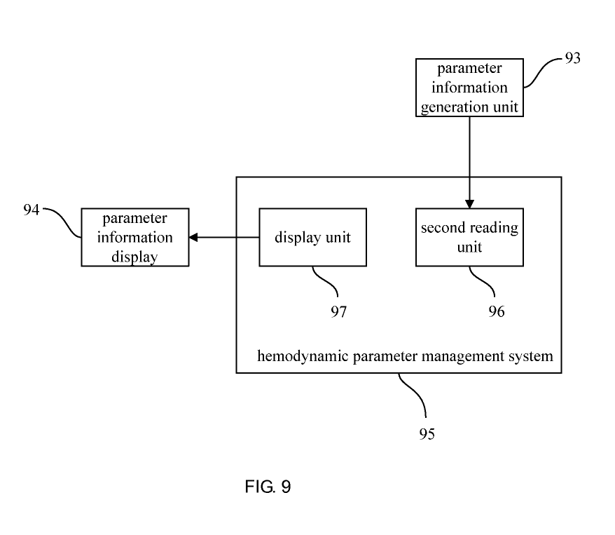

The above mentioned hemodynamic parameter management system can be applied to monitors, such as patient monitors or central monitoring systems. The structure of such monitors is shown in FIG. 9, and may include a parameter information generation unit 93 to monitor patient physiological parameter information, a parameter information display 94 to display physiological parameter information, and one of the above-mentioned hemodynamic parameter management systems 95. The parameter information generation unit 93 may collect patient physiological parameters and then obtain the parameter information after processing. The parameters may include, for example, hemodynamic parameters. The display 94 may have a functional group map displaying area. The second reading unit 96 of the hemodynamic parameter management system 95 acquires the parameter information of SHPs from the parameter information generation unit 93. The display unit 97 of the hemodynamic parameter management system 95 displays the functional map in the functional group map displaying area through the display 94.

This disclosure has been made with reference to various exemplary embodiments including the best mode. However, those skilled in the art will recognize that changes and modifications may be made to the exemplary embodiments without departing from the scope of the present disclosure. For example, various operational steps, as well as components for carrying out operational steps, may be implemented in alternate ways depending upon the particular application or in consideration of any number of cost functions associated with the operation of the system, e.g., one or more of the steps may be deleted, modified, or combined with other steps.

Additionally, as will be appreciated by one of ordinary skill in the art, principles of the present disclosure may be reflected in a computer program product on a computer-readable storage medium having computer-readable program code means embodied in the storage medium. Any tangible, non-transitory computer-readable storage medium may be utilized, including magnetic storage devices (hard disks, floppy disks, and the like), optical storage devices (CD-ROMs, DVDs, Blu-Ray discs, and the like), flash memory, and/or the like. These computer program instructions may be loaded onto a general purpose computer, special purpose computer, or other programmable data processing apparatus to produce a machine, such that the instructions that execute on the computer or other programmable data processing apparatus create means for implementing the functions specified. These computer program instructions may also be stored in a computer-readable memory that can direct a computer or other programmable data processing apparatus to function in a particular manner, such that the instructions stored in the computer-readable memory produce an article of manufacture, including implementing means that implement the function specified. The computer program instructions may also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

While the principles of this disclosure have been shown in various embodiments, many modifications of structure, arrangements, proportions, elements, materials, and components, which are particularly adapted for a specific environment and operating requirements, may be used without departing from the principles and scope of this disclosure. These and other changes or modifications are intended to be included within the scope of the present disclosure.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. As used herein, the terms “comprises,” “comprising,” and any other variation thereof, are intended to cover a non-exclusive inclusion, such that a process, a method, an article, or an apparatus that comprises a list of elements does not include only those elements but may include other elements not expressly listed or inherent to such process, method, system, article, or apparatus. Also, as used herein, the terms “coupled,” “coupling,” and any other variation thereof are intended to cover a physical connection, an electrical connection, a magnetic connection, an optical connection, a communicative connection, a functional connection, and/or any other connection.

Those having skill in the art will appreciate that many changes may be made to the details of the above-described embodiments without departing from the underlying principles of the invention. The scope of the present invention should, therefore, be determined only by the following claims.

What is claimed is:

1. An method for managing hemodynamic parameters, comprising:
loading one or more pre-created functional group templates corresponding to one or more diseases or physiological symptoms;
acquiring Selected Hemodynamic Parameters (SHPs) of functional groups from each functional group template;
acquiring parameter information of the SHPs from each real-time monitored hemodynamic parameter;
counting abnormal situations of the simultaneously abnormal SHPs in each functional group based on parameter information;
comparing the abnormal situations of the simultaneously abnormal SHPs in each functional group with predefined rules and determine a display mode of each functional group; and
displaying a functional group map according to the determined display modes.

2. The method of claim 1, wherein the parameter information comprises a parameter value; and
wherein, before counting abnormal situations, the method further comprises comparing each parameter value of the SHPs with a pre-defined normal range and determine whether and/or how much the SHPs are beyond the normal range.

3. The method of claim 1, wherein the parameter information comprises a mark used to mark whether a parameter value is abnormal.

4. The method of claim 3, wherein the predefined rules is to categorize abnormal situations of SHPs into levels according to the number of abnormal SHPs, each level corresponding to a display mode.

5. The method of claim 4, wherein, after displaying each functional group map, the method further comprises:
when an abnormal parameter value is found in a functional group, reading a monitoring time of the abnormal parameter value and displaying a message on the functional group map that changes over time according to the monitoring time.

6. The method of claim 1, wherein, after displaying each functional group map, the method further comprises:
detecting a cursor selected position at a certain functional group map; and
displaying the map segment of the time according to the cursor selected position, wherein the map segment displays parameter values of SHPs comprised by the functional group at the time.

7. The method of claim 1, wherein, after displaying each functional group map, the method further comprises:
determining a Most Typical Time (MTT) in the map by analyzing the map of a functional group, wherein the MTT is the time when there are N abnormal SHPs in the functional group and/or the time when N abnormal SHPs recover to normality, N being an integer greater or equal to 1; and
displaying all map segments at the TT, wherein the map segment displays SHP values in a functional group.

8. The method of claim 7, wherein the map segments display the SHP values at the MTT in the form of a spider vision diagram.

9. An method for managing hemodynamic parameters, comprising:
acquiring parameter information of Selected Hemodynamic Parameters (SHPs) with properties from real-time hemodynamic parameter values related to time;
calculating statistics of abnormal situations of simultaneously abnormal SHPs with the same properties;
comparing the abnormal situations of the simultaneously abnormal SHPs with same properties with predefined rules;
determining a display mode of the parameter with the properties at the monitoring time; and
taking the SHPs with the same properties as a functional group and displaying the functional group map changing over time according to the determined display mode.

10. A system for managing hemodynamic parameter, comprising:
a functional group template loading unit configured to load one or multiple predefined functional group templates;
a first reading unit configured to read Selected Hemodynamic Parameters (SHPs) in the loaded functional group template;
a second reading unit configured to acquire corresponding parameter information of the SHPs from a real-time monitored hemodynamic parameter database;
a statistical unit configured to collect simultaneously abnormal SHP situations of each functional group according to parameter information;
a display code unit configured to compare the simultaneously abnormal SHP situation of each functional group with the predefined rules, and to determine a display mode at that time; and
a display unit configured to display a map, changing over time, of each functional group according to the determined display mode.

11. The system of claim 10, wherein the predefined rules is to categorize abnormal situations of SHPs into levels according to the number of abnormal SHPs, each level corresponding to a display mode.

12. The system of claim 1, wherein the system also comprises:
a cursor detection unit configured to detect the selected position of the cursor on the function group map; and
a map segment display unit configured to display the map segment at the time corresponding to the cursor-selected position, wherein the map segment displays SHP values in a functional group at that time.

13. The system of claim 1, wherein the system further comprises:
a map analysis unit configured to analyze the map of a functional group and determine the Most Typical Time (MTT), wherein the MTT reflects the time when there are N abnormal SHPs in the functional group and/or the time when N abnormal SHPs recover to normality, N being an integer greater or equal to 1;
a map segment display unit configured to display the map segment at the MTT, wherein the map segment contains the SHPs value in the functional group at that time.

14. The system of claim 12, further comprising:
a map superimposing unit configured to receive a user-selected time and superimpose map segments in a functional group at the user-selected time in one graph.

15. The system of claim 10, further comprising:
a functional group setup unit configured to set hemodynamic parameters and/or functional group names in a functional group.

16. A system for managing hemodynamic parameter, comprising:
a property setup unit configured to set properties of hemodynamic parameters;
second reading unit configured to acquire parameter information of Selected Hemodynamic Parameters (SHPs) with properties from real-time hemodynamic parameter values related with time;
a statistical unit configured to collect abnormal situations of SHPs with the same property at the same monitoring time according to parameter information;
a display code unit configured to compare the abnormal situations of simultaneously abnormal SHPs with the same properties at the same time with predefined rules and determine a display mode of SHPs with the properties at that monitoring time;
a display unit, used to take the SHPs with the same properties as a functional group and display the functional group map which changes over time according to the determined display mode.

17. A monitoring device, comprising:
a parameter information generation unit configured to monitor patient physiological parameter information;
a parameter information display configured to display physiological parameter information, wherein the physiological parameter includes hemodynamic parameters;
a system for managing hemodynamic parameters including:
a functional group template loading unit configured to load one or multiple predefined functional group templates;
a first reading unit configured to read Selected Hemodynamic Parameters (SHPs) in the loaded functional group template;
a second reading unit configured to acquire corresponding parameter information of the SHPs from a real-time monitored hemodynamic parameter database;
a statistical unit configured to collect simultaneously abnormal SHP situations of each functional group according to parameter information;
a display code unit configured to compare the simultaneously abnormal SHP situation of each functional group with the predefined rules, and to determine a display mode at that time; and
a display unit configured to display a map, changing over time, of each functional group according to the determined display mode.
wherein the said parameter information display has a functional group map displaying area, the said second reading unit of the said hemodynamic parameter management system acquires the parameter information of SHPs from the parameter information generation unit, and the display unit of the hemodynamic parameter management system displays the functional map in the functional group map displaying area.
